(19) United States
(12) Patent Application Publication
(10) Pub. No.: US 2022/0273064 A1
(43) Pub. Date: Sep. 1, 2022

(54) TRANSPARENT HEATING ELEMENT FOR EYE PROTECTOR AND METHOD OF MANUFACTURING SAME

(71) Applicant: DAEDONG CO., LTD., Busan (KR)

(72) Inventors: Seung Taeg LEE, Sakura-shi, Chiba (JP); Hee Jung NOH, Busan (KR); Cheol Joo LEE, Busan (KR)

(21) Appl. No.: 17/612,706

(22) PCT Filed: May 9, 2020
(86) PCT No.: PCT/KR2020/006136

§ 371 Date: Nov. 19, 2021

(30) Foreign Application Priority Data
May 20, 2019 (KR) 10-2019-0058619

(51) Int. Cl. A42B 3/18; H05B 3/84

(52) U.S. Cl. CPC A42B 3/185; H05B 3/84; H05B 2203/017

(57) ABSTRACT

Proposed is a transparent heating element for an eye protector such as goggles for snowmobiling, skiing, motorcycling, and the like on which a lens is mounted, and is a manufacturing method thereof. On a surface of a transparent base that configures the lens of the eye protector, a lattice film formed of a dried material or a calcined material of a conductive ink or a conductive paste is provided. The lattice film has a line width of 2.5 μm to 20 μm, a lattice pitch of 0.1 mm to 5.0 mm, a surface resistance value of 10 Ω/cm² to 50 Ω/cm², and a light transmittance of 90% when the transparent base is excluded. An anti-fog coating treatment is performed on the opposite surface of the transparent base. The surface resistance value is partially changed by changing the lattice pitch.

### TECHNICAL FIELD

The present disclosure relates to a transparent heating element for an eye protector such as goggles for snowmobiling, goggles for skiing, goggles for motorcycling, and the like that have an anti-fog function, and relates to a method of manufacturing the transparent heating element. More particularly, the present disclosure relates to a transparent heating element for an eye protector such as goggles for snowmobiling, goggles for skiing, goggles for motorcycling and the like, in which the transparent heating element having a dried material or a calcined material of a conductive ink or a conductive paste is formed at a surface of a transparent base that constitutes a lens of the eye protector and an anti-fog coating treatment is performed at an opposite surface of the transparent base, and relates to a method of manufacturing the transparent heating element.

### BACKGROUND ART

In general, in goggles that are attached to and mounted on a helmet, when a motorcycle, a snowmobile, and the like are used in a cold region or winter, there has been a need for the goggles that use a transparent conductive film as a transparent heating element in order to melt snow on the goggles and to prevent ice formation and fogging of the goggles. In addition, the same need has been required for goggles for skiing. Currently, an Indium Tin Oxide (ITO) film that is an oxide transparent conductive material is mainly used as the transparent conductive film.

Here, as the goggles for melting snow and for preventing both the ice formation and fogging of the goggles, a configuration has been disclosed in Japanese Patent Application Publication No. 50-147192. In the configuration, a viewing plane is formed of two lenses, a transparent conductive film is formed on an entire surface of one lens, then both electrodes that are each formed in a line shape and an electrode protection plate are sequentially fixed perpendicularly on top and bottom ends of the transparent conductive film, and then the electrodes at top and bottom portions are connected with a power source via a switch. In this configuration, through a spacer formed of a material that has elasticity, heat-resistance, and cold-resistance, the other lens of the goggles is fixed to the lens on which the transparent conductive film is formed, and a sealing space is formed between the transparent conductive film and the other lens.

In a heating element of such goggles, since electricity is provided from a battery power source that is provided in a motorcycle or a snowmobile, a supplied voltage is normally limited to equal to or less than 12V. Therefore, in order to heat the transparent heating element formed of the ITO film to a temperature of 30° C. to 50° C. for purposes of melting a snow and preventing ice formation of the goggles, a resistance value equal to or less than 30 Ω/cm² to 40 Ω/cm² is required on the ITO film. In addition, since a small battery is used as a power source of the goggles for skiing, low sheet resistance value is required that is equal to or less than 10 Ω/cm². However, since a film thickness is formed to be at least 0.5 μm in order to form a low-resistance film with the ITO film, there has been a problem that transparency is lowered.

In addition, when the ITO film is used as the heating element of lens of goggles, there has been a problem that a non-uniform heating occurs. Since the ITO film is formed by performing a sputtering method in a vacuum, a film thickness of the ITO film is almost constant, so that a surface resistance value per area is approximately constant. Generally, since distances between electrodes of the goggles are different from each other depending on positions in the lens of the goggles and a center portion of the goggles covers a face by avoiding a nose area of the face, the distances between the electrodes at the top and bottom portions become short and the resistance values are lowered, so that a high current flows on the center portion of the goggles and a temperature at the center portion of the goggles becomes high. In order to maintain the minimum requirement surface temperature that is for preventing fog, and in order to ensure a battery life to be maximally lengthened, it is necessary to uniformly heat the goggles throughout entire surface of the goggles. To this end, in Japanese Patent Application Publication No. 2017-40930 (published on Feb. 23, 2017), a technology that changes the amount of current flowing depending on a distance between top and bottom electrodes by subdividing the top and bottom electrodes and realizes a uniform temperature at a heating surface exists.

Meanwhile, in Korean Patent No. 10-1857804 (registered on May 8, 2018), a polycarbonate resin sheet (thickness of 1 mm) on which an anti-fog coating treatment is performed on a surface thereof is punched to form a lens formed in a goggles shape. Then, at a distance of 20 mm from opposite ends of the lens, masking is performed at a reverse surface of the lens on which the anti-fog coating treatment is performed, and sputtering of ITO is performed and the ITO transparent conductive film having a film thickness of 190 nm and a surface resistance value of 30 Ω/cm² is formed. A fog-resistant structure formed in the manner as described above and a protective device for eyes are disclosed in Korean Patent No. 10-1857804.

### DISCLOSURE

### Technical Problem

As described above, In Japanese Patent Application Publication No. 50-147192 and in Korean Patent No. 10-1857804, when a heating element having a low-resistance value equal to or less than 40 Ω/cm² and formed of a transparent conductive film is formed on an ITO film, light transmittance becomes equal to or less than 90%, so that there is a problem in using the heating element in goggles in which visibility is an important factor. In addition, a transparent base such as Polyethylene terephthalate (PET), Polyethylene naphthalate (PEN), or the like is used in the goggles. Therefore, in the viewpoint of lightweightness and flexibility, there is a need for a material having flexibility against external stress. However, since the ITO film is an oxide film, the ITO film has hard and fragile characteristics, so that there is a problem in that cracking may occur when a strong shock from outside is applied to the ITO film. Further, since a large vacuum device is required to form the ITO film, there is a problem that both the procedure and the manufacturing cost for forming the transparent film are increased.

In addition, in order to change the amount of current depending on a distance between the top and bottom electrodes by subdividing the top and bottom electrodes, it is necessary to apply a new control element for controlling a power source. Further, difficulties such as a complexity of a circuit occur, so that there are problems that performance of a required function is degraded and manufacturing cost becomes expensive.

As a result of conducting a considerable review so as to solve the problems of the ITO film that is described above, a technical objective of the present disclosure is to provide a transparent heating element for an eye protector and a method of manufacturing the transparent heating element, in which a fine wiring lattice film is formed on an outer surface of a lens by performing a printing method that uses a conductive ink or a conductive paste instead of the ITO film, and in which heating is performed by allowing electricity to flow on a lattice film pattern, thereby replacing the ITO film with the transparent heating element.

### Technical Solution

In order to achieve the above objective, according to one aspect of the present disclosure, there is provided a transparent heating element for an eye protector, the transparent heating element including: a lattice film formed of a dried material or a calcined material of a conductive ink or a conductive paste and formed at at least one surface of a transparent base, the lattice film being configured such that a line width thereof is 2.5 μm to 20 μm, a lattice pitch thereof is 0.1 mm to 5.0 mm, a surface resistance value thereof is 10 Ω/cm² to 50 Ω/cm², and a light transmittance thereof excluding a portion of the transparent base is at least 90%, wherein, in the lattice film, the surface resistance value of the surface on which the dried material or the calcined material of the conductive ink or the conductive paste is formed is partially changed by changing the lattice pitch.

In addition, according to the transparent heating element for the eye protector of the present disclosure, the transparent base may be an organic film or a glass base.

In addition, according to the transparent heating element for the eye protector of the present disclosure, the transparent heating element may be formed at one surface of the transparent base, and an anti-fog coating treatment may be performed on opposite surface of the surface on which the transparent heating element is formed.

In addition, according to the transparent heating element for the eye protector of the present disclosure, the eye protector may be goggles for snowmobiling, goggles for skiing, or goggles for motorcycling.

In addition, in order to achieve the above objective, according to one aspect of the present disclosure, there is provided a method of manufacturing the transparent heating element for an eye protector, the method including: forming a lattice film of a conductive ink or a conductive paste by performing a screen printing method, a gravure printing method, a gravure offset printing method, a gravure reverse printing method, an imprint printing method, or an inkjet printing method.

### Advantageous Effects

As described above, according to the transparent heating element for an eye protector and a method of manufacturing the transparent heating element according to the present disclosure, visibility of goggles having the transparent base may be secured, and the transparent heating element having the dried material or the calcined material of the conductive ink or the conductive paste that can be formed to have the desired resistance value may be provided.

According to the present disclosure, the visibility of the goggles having the transparent base may be secured, and the transparent heating element having the dried material or the calcined material of the conductive ink or the conductive paste that can be formed to have the desired resistance value may be easily manufactured.

According to the present disclosure, the visibility of the goggles having the transparent base may be secured. Further, by the transparent heating element having the dried material or the calcined material of the conductive ink or the conductive paste that can be formed to have the desired resistance value, goggles for snowmobiling, goggles for skiing, goggles for motorcycling, and the like that are capable of preventing fogging may be provided.

### DESCRIPTION OF DRAWINGS

FIG. 1 is a schematic view illustrating a lattice pattern forming a rectilinear lattice film.

FIG. 2 is a schematic view illustrating a shape of a lens of goggles used in Embodiment 1.

FIG. 3 is a schematic view illustrating a shape of a lens of goggles used in Embodiment 4.

### MODE FOR INVENTION

The above and other objectives and new features of the present disclosure will become more apparent from the description of the present specification and the accompanying drawings.

Hereinafter, an embodiment of the present disclosure will be described with reference to the drawings.

[Transparent Heating Element]

According to a transparent heating element (hereinafter, referred to as “transparent heating element”) for an eye protector of the present disclosure, a lattice film formed of a dried material or a calcined material of a conductive ink or a conductive paste and having a line width of 2.5 μm to 20 μm and having a lattice pitch of 0.1 mm to 5.0 mm is formed on at least one surface of a transparent base, a surface resistance value of the lattice film is 10 Ω/cm² to 50 Ω/cm², and a light transmittance of the lattice film at a portion excluding the transparent base is at least 90%. Further, in the lattice film, the surface resistance value at the surface formed of the dried material or the calcined material of the conductive ink or the conductive paste is partially changed resulting from a change of the lattice pitch. In the viewpoint of simplicity of manufacturing the lattice film and in the viewpoint of manufacturing cost of the lattice film, the dried material or the calcined material of the conductive ink or the conductive paste is used to form the lattice film. Here, a rectilinear lattice refers to a lattice having an intersection angle of 45 degrees to 90 degrees between a vertical line and a horizontal line.

In addition, the term “the dried material or the calcined material of the conductive ink or the conductive paste” used in the present disclosure corresponds to “Product by Process Claim”. However, in order to distinguish “the dried material or the calcined material of the conductive ink or the conductive paste” from an Indium Tin Oxide (ITO) film that is in the conventional technology, “the dried material or the calcined material of the conductive ink or the conductive paste” is used as a conductive film.

In the transparent heating element, when the surface resistance value of the surface on which the dried material or the calcined material of the conductive ink or the conductive paste is formed is changed resulting from the change of the lattice pitch, the desired resistance value can be achieved, so that it is preferable to use the transparent heating element.

In the viewpoint of increasing reliability of an anti-fog function, in this transparent heating element, it is preferable that the transparent heating element is formed at one surface of the transparent base and an anti-fog coating treatment is performed on a surface opposite to the surface on which the transparent heating element is formed.

In a conductive lattice film (hereinafter, referred to as “lattice film”) formed of the dried material or the calcined material of the conductive ink or the conductive paste, the inventor of the present disclosure has found that when a width of a wiring configuring the lattice film is set to be equal to or less than 20 μm, the lattice film does not deteriorate in visibility and does not block or interrupt the sight of a user who wears an eye protector in which the lattice film is used. In addition, the inventor of the present disclosure has found that an actual light transmittance (a light transmittance including a light transmittance of the transparent base) can be set to at least 85% by setting the wiring width to be equal to or less than 20 μm and by setting a wiring pitch to be 0.1 mm to 5.0 mm, and the inventor of the present disclosure has found that the surface resistance value of the lattice film can be controlled in the desired range by changing the wiring width of the lattice film and the wiring pitch of the lattice film. As a result, by widening the wiring pitch depending on a distance at a portion where the distance between a top electrode and a bottom electrode is short and by increasing the resistance value depending on a surface area, temperature may be substantially and uniformly controlled by only connecting a single power source to the top and bottom electrodes.

Meanwhile, in the viewpoint of securing a conductivity and in the viewpoint of applying a convenient printing method that is available, the wiring width of the lattice film is preferable to be at least 2.5 μm. In the viewpoint of the light transmittance of the transparent heating element, the lattice pitch of the lattice film is preferably at least 0.1 mm. Here, it is preferable that the wiring width is 2.5 μm to 20 μm. Further, it is more preferable that the wiring width is 3.0 μm to 10 μm. It is preferable that the lattice pitch is 0.1 mm to 5.0 mm. Further, it is more preferable that the lattice pitch is 0.5 mm to 3.0 mm.

As a conductive ink or a conductive paste used in the dried material or the calcined material of the conductive ink or the conductive paste to form the conductive film, it is preferable to use a conductive ink or a conductive paste containing a silver nano ink or a silver nanoparticle paste as a main material. Compared to an ITO oxide film transparent conductive material having a specific resistance value of 5×10⁻⁴ Ω·cm and conventionally used as a transparent heating element, the silver nano ink or the silver nanoparticle paste can be formed into a film by performing a printing method in the atmosphere. Further, by performing a calcinating to silver nano ink or the silver nanoparticle paste at a temperature of 130 C after the silver nano ink or the silver nanoparticle paste is formed into a conductive film by performing a drying, a specific resistance value thereof can be set to about equal to or less than 1/100 of the specific resistance value of the ITO oxide film transparent conductive material, and an actual light transmittance (a light transmittance including a light transmittance of the transparent base) can be set to at least 85% since the conductive film is the lattice film.

In addition, since the conductive film is formed of the lattice film having very fine lines, the conductive film is resistant to twisting and bending. Further, since the conductive film is formed by performing the printing method, a functionality thereof may be improved, and a significant portion of manufacturing cost may be reduced since the large vacuum device is not required and there is no need for a wait time that is due to a vacuum process.

In goggles that have different shapes depending on the shape of a person's face, a distance between the top and bottom electrodes is different depending on a place where the goggles are used. Therefore, in a heating element, such as the ITO film, having a uniform resistance value, when a constant voltage is applied between the top and bottom electrodes, ununiformity of heating of the film occurs, so that life of a battery is shortened when a temperature of the goggles is maintained below a dew point so as to prevent fog from being formed through a front surface of the goggles.

In the lattice film of the present disclosure, since the surface resistance value can be changed depending on a place by changing a printed pitch of the lattice depending on the place, there is no need to divide the top and bottom electrodes by a region and there is no need to control and supply a power required for the region. Further, an entire viewing plane of the goggles can be heated to a uniform temperature, and a configuration of the goggles can be simplified. Therefore, the manufacturing cost may be highly reduced, and the functionality of the goggles may be improved.

There is no specific limit to the transparent base used in the transparent heating element, but an organic film or a glass base is used in the transparent base, and it is preferable to use the organic film. For an example, a film formed of a material such as Polyethylene terephthalate (PET), Polyethylene naphthalate (PEN), Polycarbonate (PC), and so on are used as the organic film. In addition, even when the glass base is used in the transparent base, the dried material of the calcined material of the conductive ink or the conductive paste that is formed of a metal is more resistant to external shock than the ITO film that is formed of a ceramic material.

In the viewpoint of the convenience of manufacturing and in the viewpoint of manufacturing cost, for example, the lattice film may be formed by performing a screen printing method, a gravure printing method, a gravure offset printing method, a gravure reverse printing method, an imprint printing method, or an inkjet printing method. The printing method will be described later.

The required surface resistance value for the transparent heating element is preferable to be 10 Ω/cm² to 50 Ω/cm², and is more preferable to be 20 Ω/cm² to 40 Ω/cm². Further, in the light transmittance at the wavelength of 550 nm excluding a transmittance of a base film, the light transmittance is preferable to be at least 90%, and is more preferable to be 92% to 98%.

[Method of Manufacturing Transparent Heating Element]

In a method of manufacturing the transparent heating element of the present disclosure, the screen printing method, the gravure printing method, the gravure offset printing method, the gravure reverse printing method, the imprint printing method, or the inkjet printing method may be applied to a method of forming the lattice film of the conductive ink or the conductive paste. In the viewpoint of degrading of conductivity of the lattice film after the lattice film formed of various printing methods is dried, it is preferable to perform firing at a temperature equal to or less than a heat-resistance temperature of the transparent base that is a substrate. In addition, the lattice film is formed on the transparent base. For example, the lattice film is formed by performing the printing method, then the lattice film is fired at a temperature equal to or less than a heat-resistance temperature of a substrate film, then a pair of electrode patterns connected to the top and bottom of the lattice film or the left and right of the lattice film is formed by performing the same printing method, and then the pair of electrode patterns is fired in the same method. For the purpose of protecting the lattice film, it is possible to coat the lattice film with a transparent resin film by printing the transparent resin film on the lattice film. Hereinafter, an example of forming the lattice film and an example of forming electrodes that are connected to the lattice film will be described.

In the gravure offset printing method, it is possible to use a conventional gravure offset printing device. A silver nanoparticle ink is injected at a concaved portion of a gravure roll that is provided with a lattice film pattern and an electrode pattern, then the silver nanoparticle ink remaining on surfaces of the lattice film pattern and the electrode pattern is swept off and removed by using a scraper, and then the lattice pattern is copied to a blanket roll by pressing the gravure roll onto the blanket roll. Next, the pattern on the blanket roll is copied to the transparent base that is sucked and fixed at a reduced-pressure suction stage. After the transparent base that is printed is dried, the transparent base is fired in a furnace in which a temperature is set to 130° C. After continuous printing on the transparent base, the results may be fired together.

In the formation of the electrodes that are connected to the lattice pattern, it is preferable to form the electrodes by using a screen printing device using a silver nanoparticle paste, after the lattice film pattern is formed by using the gravure offset printing device. In this case, after the lattice film pattern is formed, the transparent base that is dried and fired is set on the reduced-pressure suction stage of the screen printing device so that the transparent base is sucked and fixed at the reduced-pressure suction stage. On the transparent base, for example, a screen plate for the electrode pattern is set to be spaced apart at intervals of several millimeters, then the silver nanoparticle paste is set on the screen plate, then the silver nanoparticle paste is spread by putting a squeegee on the screen plate, and then the printing is performed by moving a substrate table. Next, the transparent base substrate that is printed is extracted by lifting the squeegee and by moving a substrate stage, then the transparent base substrate is dried and is fired in the furnace in which the temperature is set to 130° C. Then, a substrate on which another lattice film pattern is formed is set on the reduced-pressure suction stage, and the printing is repeated in the same manner. After the printing is continuously performed, the results may be fired together. As an embodiment that will be described later, the lattice pattern may be formed by performing the screen printing.

The goggles using the transparent heating element of the present disclosure are capable of being applied to goggles for snowmobiling, goggles for skiing, and goggles for motorcycling. Further, the goggles can be used throughout the year, and are particularly suitable for use in cold regions or winter where a function capable of preventing the formation of dew or fog is required.

[Assembly of Goggles with Transparent Heating Element]

Hereinafter, a method of assembling goggles having a transparent heating element will be described.

First, a transparent heating element is prepared. FIG. 2 is a schematic view illustrating a shape of a lens of goggles used in Embodiment 1 that will be described later. In the transparent heating element, a lattice film 2, a top end electrode 10, and a bottom end electrode 20 are formed on a PET film (the PET film becomes an inner lens of the goggles), and the transparent heating element is cut into a target size of a lens of goggles. In the transparent heating element, holes 11 and 21 that have diameters of 4 mm and that are for connecting connection terminals are respectively formed in a left end of the top end electrode 10 and a right end of the bottom end electrode 20. In order to separately fix the transparent heating element and the connection terminals, four sheets of SUS plates (diameter: 8 mm) and two pairs of screws and nuts are prepared.

A hole having a size that is almost the same as the hole for connecting the electrode is formed in both four sheets of the SUS plates and the connection terminals. In addition, a lead wire is respectively mounted on the connection terminals in advance. After arranging the SUS plate, the connection terminal, the PET film, the electrode, and the SUS plate in order, the screw is inserted into the hole and fixed with the nut. Then, the electrode and the SUS plate at the electrode are covered with an electrode protective agent, and are separated.

This process is performed on the top electrode and the bottom electrode. Separately, a material for a spacer is prepared and a PET film that becomes an outer lens of the goggles is prepared. As a material of the spacer, a material having elasticity, cold-resistance, and heat-resistance is preferable to be used. After the process described above is performed, the PET film that becomes the inner lens of the goggles, the electrode, the spacer, the PET film that becomes the outer lens of the goggles are arrange in order and are fixed to the goggles, so that the goggles having the transparent heating element is assembled. Here, at the spacer, a sealing space is formed between the PET film that becomes the inner lens and the PET film that becomes the outer lens, so that the transparent heating element is protected by the PET film that becomes the outer lens.

### Embodiment 1

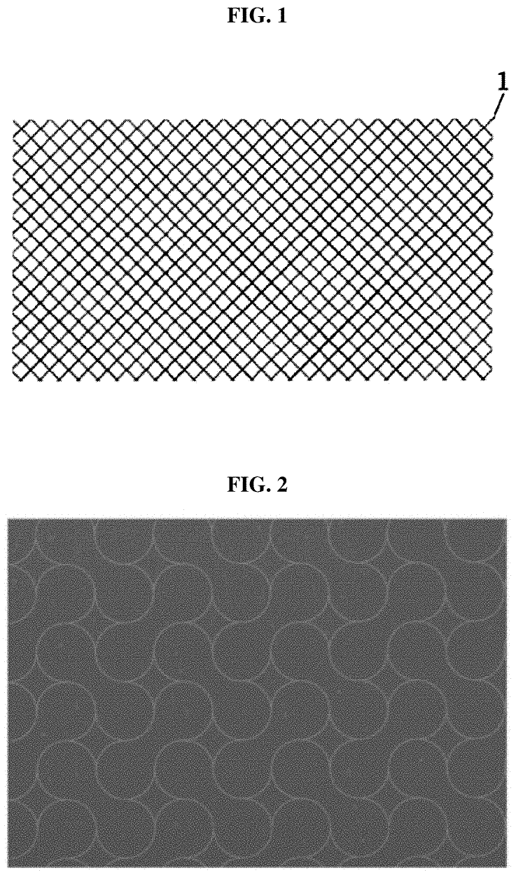

On a PET film Cosmoshine A4300 (film thickness: 250 μm, size: 130 mm×370 mm, adhesion layers are attached to opposite surfaces thereof, and an anti-fog coating film is formed at one surface thereof) manufactured by Toyobo Co., Ltd., by performing a screen printing method by using both a silver paste that is a product of Daicel Co., Ltd., (product name: Picosil, solid content concentration: 65 wt %, viscosity: 10,000 mPas) and a screen plate that is a product of Murakami Co., Ltd., (product name: Hi convi 550 (360) CAL-ER44003, thickness: 40 μm), a lattice film pattern was printed on a surface on which the anti-fog coating film is not formed. A lattice line width was 20 μm, a lattice pitch was 1 mm, an intersection angle was 90 degrees, and lattice lines were inclined to 45 degrees with respect to a direction from a top electrode to a bottom electrode. In FIG. 1, a schematic view of a lattice pattern 1 provided with a rectilinear lattice film is illustrated.

black solid lines in FIG. 1 are lines forming the lattice film. For a vertical width of the lattice pattern 1, a uniform pattern was used through a center portion and left and right end portions thereof. In the same manner, printing was repetitively performed on the PET film at intervals of about 15 seconds, and 50 sheets were printed in about 13 minutes. After finishing the printing, the 50 sheets were dried. Then, the 50 sheets were inserted into a furnace in which a temperature is fixed at 130° C., and were fired for 30 minutes, and then a firing of the printed lattice patterns were completed. Next, by using the same silver paste that is the screen plate product of Murakami Co., Ltd., (660×660 CAL400-φ23, thickness: 40 μm), a top electrode and a bottom electrode were respectively printed at a top end and a bottom end of the lattice pattern of a base in which the firing of the lattice pattern is completed by performing the screen printing method.

An electrode width was 6 mm and an electrode thickness was 5 μm. In the same manner, printing was repetitively performed on the PET film at intervals of about 15 seconds, and 50 sheets were printed in about 13 minutes. After finishing the printing, the 50 sheets were dried. Then, the 50 sheets were inserted into a furnace in which a temperature is fixed at 130° C., and were fired for 30 minutes, and then a firing of the printed electrode patterns were completed.

After performing the firing, the thickness of the lattice pattern was about 0.6 μm, and the thickness of the electrode pattern was about 5 μm. Although there is a difference in the resistance values between the 50 sheets of the top and bottom electrodes, the resistance values were included between 3.0 Ω to 3.5 Ω, and an average value of the resistance values was about 3.2 Ω and an average value of surface resistance values was about 10 Ω/cm². One of the sheets was arbitrarily selected and a total light transmittance was measured. As a result, the total light transmittance was about 95% when the light transmittance of a PET substrate is excluded. Since the lattice film does not cover an entire surface of a lens, a haze value of the lattice film was the same as a haze value of the PET substrate (the total light transmittance of the PET substrate A4300 having the thickness of 250 μm: 92.3%, haze value: 0.9%).

One sheet is arbitrarily selected from these 50 sheets of the transparent heating elements formed of the silver lattice film that is printed, dried, and fired, and is cut to the desired size of lens of goggles. That is, a schematic view of goggles having a conventional shape (size is 100 mm in a vertical direction×340 mm in a horizontal direction) is illustrated in FIG. 2. Holes 11 and 21 for inserting connection terminals were respectively formed in end portions of the electrodes 10 and 20 that are respectively positioned at the top and bottom portions of the transparent heating element. By using the same method that is described in [Assembly of Goggles with Transparent Heating Element], the goggles provided with the transparent heating element were assembled. A target reaching temperature of the goggles was 45° C.

A power source is connected to the goggles and a voltage of 5.5V was applied to the goggles so as to heat the goggles, and results were shown in Table 1. A non-contact type radiation thermometer (product name: fixed density radiation thermometer manufactured by Horiba Ltd., type: IT545S) was used to measure a temperature, and temperatures of several positions inside the goggles were measured. As a result, since a large difference in temperatures according to measurement positions was not identified, temperatures at the measurement position of a center portion of the goggles from left and right positions and from top and bottom positions of the goggles were recorded. After four minutes, the temperature rose to 42.5 ° C., and, after eight minutes, the temperature reached about 45° C. that is the target reaching temperature.

Under an environment in which an outside air temperature was 0° C. and a humidity was 40%, a helmet for motorcycling on which the goggles were mounted was worn on a head, and the transparent conductive film was conductively heated by connecting the power source. As a result, when in a state in which the goggles were mounted on the face, there was no fog on a surface of the lens, and the lines of the lattice pattern did not interfere with the sight.

TABLE 1

| Elapsed time | Voltage | Current | Power | V/A | Temperature |
|---|---|---|---|---|---|
| (minutes) | (V) | (A) | (W) | (Ω) | (° C.) |
| 4 | 5.5 | 1.65 | 9.1 | 3.3 | 42.5 |
| 6 | 5.5 | 1.63 | 9.0 | 3.4 | 43.5 |
| 8 | 5.5 | 1.62 | 8.9 | 3.4 | 44.6 |
| 10 | 5.5 | 1.61 | 8.9 | 3.47 | 44.8 |
| 12 | 5.5 | 1.58 | 8.7 | 3.5 | 44.3 |
| 16 | 5.5 | 1.57 | 8.6 | 3.5 | 44.9 |

### Embodiment 2

On a PET film Cosmoshine A4300 (film thickness: 250 μm, size: 130 mm×370 mm, adhesion layers are attached to opposite surfaces thereof, and an anti-fog coating film is formed at one surface thereof) manufactured by Toyobo Co., Ltd., by performing a gravure offset printing method by using both a silver paste that is a product of Fujikura Kasei Co., Ltd., (product name: DOTITE XA-3609, solid content concentration: 72 wt %, viscosity: 25,000 mPas) and an electroforming gravure concaved plate that is a product of Athene Co., Ltd., a lattice film pattern was printed on a surface on which the anti-fog coating film is not formed. The lattice pattern was the same as Embodiment 1, but the lattice line width was 5 μm, the lattice pitch was 1 mm, the intersection angle was 90 degrees, and the lattice lines were inclined to 45 degrees with respect to the direction from the top electrode to the bottom electrode.

For the vertical width of the lattice pattern, the uniform pattern was used through a center portion and left and right end portions thereof. In the same manner, printing was repetitively performed on the PET film at intervals of about 15 seconds, and 50 sheets were printed in about 13 minutes. After finishing the printing, the 50 sheets were dried. Then, the 50 sheets were inserted into a furnace in which a temperature is fixed at 130° C., and were fired for 30 minutes, and then a firing of the printed lattice patterns were completed.

Next, by using both the silver paste that is the product of Daicel Co., Ltd., (product name: Picosil, solid content concentration: 65 wt %, viscosity: 10,000 mPas) and the screen plate that is the product of Murakami Co., Ltd., (product name: Hi convi 550 (360) CAL-ER440φ13, thickness: 40 μm), the top electrode and the bottom electrode were printed at the top end and the bottom end of the lattice pattern of the base in which the firing of the lattice pattern is completed by performing the screen printing method. The electrode width was 6 mm and the electrode thickness was 5 μm. In the same manner, printing was repetitively performed on the PET film at intervals of about 15 seconds, and 50 sheets were printed in about 13 minutes.

After finishing the printing, the 50 sheets were dried. Then, the 50 sheets were inserted into a furnace in which a temperature is fixed at 130° C., and were fired for minutes, and then a firing of the printed electrode patterns were completed. After performing the firing, the thickness of the lattice pattern was about 0.6 μm, and the thickness of the electrode pattern was about 5 μm. Although there is a difference in the resistance values between the 50 sheets of the top and bottom electrodes, the resistance values were included between 4.8 Ω to 5.2 Ω and the average value of the resistance values was about 5.0 Ω and the average value of surface resistance values were about 15 Ω/cm².

One of the sheets was arbitrarily selected and a total light transmittance was measured. As a result, the total light transmittance was about at least 90% when the light transmittance of the PET substrate is excluded. Since the lattice film does not cover the entire surface of the lens, the haze value of the lattice film was the same as the haze value of the PET substrate (the total light transmittance of the PET substrate A4300 having the thickness of 250 μm: 92.3%, haze value: 0.9%).

In the same manner that is used in Embodiment 1, goggles for motorcycling were manufactured by arbitrarily selecting one sheet from these 50 sheets of the transparent heating elements formed of the silver lattice film that is printed, dried, and fired. The target reaching temperature of the goggles was 53° C.

The power source is connected to the goggles and the voltage of 8.5V was applied to the goggles so as to heat the goggles, and results were shown in Table 2. The non-contact type radiation thermometer (product name: fixed density radiation thermometer manufactured by Horiba Ltd., type: IT545S) was used to measure a temperature, and temperatures of several positions inside the goggles were measured. As a result, since a large difference in temperatures according to measurement positions was not identified, temperatures at the measurement position of the center portion of the goggles from left and right positions and from top and bottom positions of the goggles were recorded. After four minutes, the temperature rose to 50.5 ° C., and, after eight minutes, the temperature reached about 53° C. that is the target reaching temperature.

Under the environment in which an outside air temperature was 0° C. and a humidity was 40%, a helmet for motorcycling on which the goggles were mounted was worn on a head, and the transparent conductive film was conductively heated by connecting the power source. As a result, when in a state in which the goggles were mounted on a face, there was no fog on a surface of lens, and the lines of the lattice pattern did not interfere with the sight.

TABLE 2

| Elapsed time | Voltage | Current | Power | V/A | Temperature |
|---|---|---|---|---|---|
| (minutes) | (V) | (A) | (W) | (Ω) | (° C.) |
| 4 | 8.5 | 1.70 | 14.5 | 5.0 | 50.5 |
| 6 | 8.5 | 1.67 | 14.2 | 5.1 | 51.6 |
| 8 | 8.5 | 1.65 | 14.0 | 5.2 | 52.7 |
| 10 | 8.5 | 1.64 | 13.9 | 5.2 | 53.2 |
| 12 | 8.5 | 1.62 | 13.8 | 5.2 | 53.1 |
| 16 | 8.5 | 1.61 | 13.7 | 5.2 | 54.1 |

### Embodiment 3

Except that the pitch having the lattice line width of 5 μm was changed from 100 μm to 200 μm, in the same condition as Embodiment 2, 50 sheets of transparent heating elements were printed, dried, and fired. Although there is a difference in the resistance values between the 50 sheets of the top and bottom electrodes, the resistance values were included between 10.0 Ω to 12 Ω and the average value of the resistance values was about 11.0 Ω and the average value of surface resistance values was about 33 Ω/cm².

One of the sheets was arbitrarily selected and the total light transmittance was measured. As a result, the total light transmittance was about 94% when the light transmittance of the PET substrate is excluded. Since the lattice film does not cover the entire surface of the lens, the haze value of the lattice film was the same as the haze value of the PET substrate (the total light transmittance of the PET substrate A4300 having the thickness of 250 μm: 92.3%, haze value: 0.9%).

In the same manner that is used in Embodiment 1, goggles for snowmobiling were manufactured by arbitrarily selecting one sheet from these 50 sheets of the transparent heating elements formed of the silver lattice film that is printed, dried, and fired. The target reaching temperature of the goggles was 50° C.

The power source is connected to the goggles and the voltage of 12V was applied to the goggles so as to heat the goggles, and results were shown in Table 3. The non-contact type radiation thermometer (product name: fixed density radiation thermometer manufactured by Horiba Ltd., type: IT545S) was used to measure a temperature, and temperatures of several positions inside the goggles were measured. As a result, since a large difference in temperatures according to measurement positions was not identified, temperatures at the measurement position of the center portion of the goggles from the left and right positions and from the top and bottom positions of the goggles were recorded.

After four minutes, the temperature rose to 47.5 ° C., and, after eight minutes, the temperature reached about 50° C. that is the target temperature. Under the environment in which an outside air temperature was −10° C. and a humidity was 40%, a helmet for snowmobiling on which the goggles were mounted was worn on a head, and the transparent conductive film was conductively heated by connecting the power source. As a result, when in a state in which the goggles were mounted on a face, there was no fog on the surface of lens, and the lines of the lattice pattern did not interfere with the sight.

TABLE 3

| Elapsed time | Voltage | Current | Power | V/A | Temperature |
|---|---|---|---|---|---|
| (minutes) | (V) | (A) | (W) | (Ω) | (° C.) |
| 4 | 12.0 | 1.09 | 13.1 | 11.0 | 47.5 |
| 6 | 12.0 | 1.07 | 12.8 | 11.2 | 48.6 |
| 8 | 12.0 | 1.06 | 12.7 | 11.3 | 49.7 |
| 10 | 12.0 | 1.04 | 12.5 | 11.5 | 49.3 |
| 12 | 12.0 | 1.02 | 12.2 | 11.8 | 49.6 |
| 16 | 12.0 | 0.99 | 11.9 | 12.1 | 49.5 |

### Embodiment 4

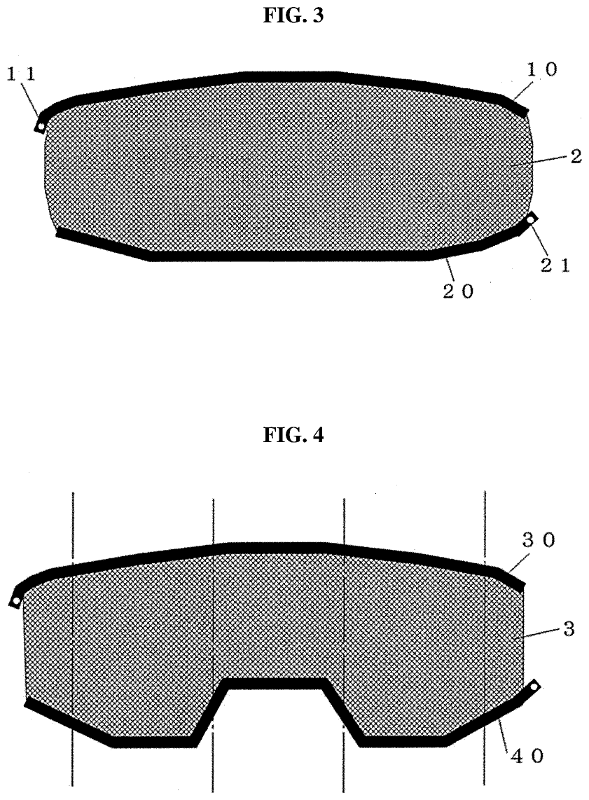

Except that a shape and a pitch of the lattice film are changed depending on a place for goggles for snowmobiling with a new design in which vertical widths of the electrodes at the center portion and at the end portions thereof were narrowed, in the same condition as Embodiment 3, 50 sheets of transparent heating elements were printed, dried, and fired. Specifically, the width of the lattice film was set to 5 μm, the pitch of both the center portion and the end portions where the distance between the top and bottom electrodes was short was set to 260 μm, and the pitch of other portions was set to 200 μm. FIG. 3 is a schematic view illustrating a shape of goggles that is used. Here, dotted lines refers to boundary lines where the pitch is changed.

In the goggles used in FIG. 3, the vertical distance between electrodes 30 and 40 at the center portion of the goggles and the vertical distance between the electrodes 30 and 40 at the end portions of the goggles are shorter than vertical distances of other portions of the goggles. As described above, since the distance between the electrodes 30 and 40 is changed depending on a position on the goggles, a boundary portion where the pitch of the lattice film 3 is changed is set to a center boundary portion. Although there is a difference in the resistance values between the 50 sheets of the top and bottom electrodes, the resistance values were included between 9.5 Ω to 12.5 Ω and the average value of the resistance values was about 11.0 Ω and the average value of surface resistance values was about 37 Ω/cm². One of the sheets was arbitrarily selected and a total light transmittance was measured. As a result, the total light transmittance was about 94% at the portion where the pitch was 200 μm when the light transmittance of the PET substrate is excluded, and the total light transmittance was about 95% at the portion where the pitch was 260 μm when the light transmittance of the PET substrate is excluded.

Since the lattice film does not cover the entire surface of the lens, the haze value of the lattice film was the same as the haze value of the PET substrate (the total light transmittance of the PET substrate A4300 having the thickness of 250 μm: 92.3%, haze value: 0.9%).

In the same manner that is used in Embodiment 3, goggles for snowmobiling were manufactured by arbitrarily selecting one sheet from these 50 sheets of the transparent heating elements formed of the silver lattice film that is printed, dried, and fired. The target reaching temperature of the goggles was 50° C.

The power source is connected to the goggles and the voltage of 12V was applied to the goggles so as to heat the goggles, and results were shown in Table 4. The non-contact type radiation thermometer (product name: fixed density radiation thermometer manufactured by Horiba Ltd., type: IT545S) was used to measure a temperature, and temperatures of several positions inside the goggles were measured. As a result, since a large difference in temperatures according to measurement positions was not identified, temperatures at the measurement position of the center portion of the goggles from the left and right positions and from the top and bottom positions of the goggles were recorded.

In each positions, the temperature rose to 47.6 ° C. to 48.3° C. after four minutes, and, after eight minutes, the temperature reached about 50° C. that is the target reaching temperature.

As a result, even if the distances between the top and bottom electrodes are different, there was no large difference in heating temperatures at each portion since the lattice pitch of the lattice film was changed at each portion where the distances between the electrodes are different.

Under the environment in which an outside air temperature was −10° C. and a humidity was 40%, a helmet for snowmobiling on which the goggles were mounted was worn on a head, and the transparent conductive film was conductively heated by connecting the power source. As a result, when in a state in which the goggles were mounted on a face, there was no fog on the surface of the lens, and the lines of the lattice pattern did not interfere with the sight.

TABLE 4

| | | | | | Temperature (° C.) | | | | |
|---|---|---|---|---|---|---|---|---|---|
| | | | | | | In front | In front | | |
| Elapsed time | Voltage | Current | Power | V/A | Center | of left | of right | Left | Right |
| (minutes) | (V) | (A) | (W) | (Ω) | portion | eye | eye | end | end |
| 4 | 12.0 | 1.12 | 13.4 | 10.7 | 48.3 | 48.1 | 47.9 | 47.6 | 47.4 |
| 6 | 12.0 | 1.11 | 13.3 | 10.8 | 49.4 | 49.2 | 48.7 | 48.3 | 48.6 |
| 8 | 12.0 | 1.09 | 13.1 | 11.0 | 50.7 | 50.3 | 49.8 | 49.5 | 49.4 |
| 10 | 12.0 | 1.07 | 12.8 | 11.2 | 50.4 | 50.1 | 49.6 | 49.2 | 49.3 |
| 12 | 12.0 | 1.05 | 12.6 | 11.4 | 50.3 | 49.8 | 49.4 | 49.1 | 49.2 |
| 16 | 12.0 | 1.04 | 12.5 | 11.5 | 50.5 | 50.2 | 49.8 | 49.1 | 49.3 |

### Embodiment 5

The shape and the pitch of the lattice film are not changed depending on a place for the goggles for snowmobiling with the new design in which vertical widths of the electrodes at the center portion and at the end portions thereof were narrowed, and the pitch was fixed at 200 μm, then 50 sheets of transparent heating elements were printed, dried, and fired in the same condition as Embodiment 4. Although there is a difference in the resistance values between the 50 sheets of the top and bottom electrodes, the resistance values were included between 8.5 Ω to 11 Ω and the average value of the resistance values was about 10.0 Ω and the average value of the surface resistance values was about 33 Ω/cm².

In the same manner that is used in Embodiment 4, goggles for snowmobiling were manufactured by arbitrarily selecting one sheet from these 50 sheets of the transparent heating elements formed of the silver lattice film that is printed, dried, and fired. The target reaching temperature of the goggles was 60° C.

The power source is connected to the goggles and the voltage of 12V was applied to the goggles so as to heat the goggles, and results were shown in Table 5. In each positions, the temperature rose to 48.1° C. to 58.2° C. after four minutes, and, after eight minutes, the temperatures at the center portion and the end portions reached about 60° C. that is the target reaching temperature. As a result, at positions where the distances are different from each other, a difference of about 10° C. in target reaching temperature is generated, and the temperature is unnecessarily increased, and it has been confirmed that unnecessarily high power consumption is used accordingly.

Under the environment in which an outside air temperature was −10° C. and a humidity was 40%, a helmet for snowmobiling on which the goggles were mounted was worn on a head, and the transparent conductive film was conductively heated by connecting the power source. As a result, when in a state in which the goggles were mounted on a face, there was no fog on the surface of the lens, and the lines of the lattice pattern did not interfere with the sight.

TABLE 5

| | | | | | Temperature (° C.) | | | | |
|---|---|---|---|---|---|---|---|---|---|
| | | | | | | In front | In front | | |
| Elapsed time | Voltage | Current | Power | V/A | Center | of left | of right | Left | Right |
| (minutes) | (V) | (A) | (W) | (Ω) | portion | eye | eye | end | end |
| 4 | 12.0 | 1.21 | 14.5 | 9.9 | 58.2 | 48.2 | 48.1 | 57.6 | 57.8 |
| 6 | 12.0 | 1.20 | 14.4 | 10.0 | 59.5 | 49.3 | 48.8 | 58.3 | 58.5 |
| 8 | 12.0 | 1.19 | 14.3 | 10.1 | 60.5 | 50.4 | 50.1 | 59.5 | 59.3 |
| 10 | 12.0 | 1.17 | 14.0 | 10.3 | 60.2 | 50.3 | 49.7 | 59.2 | 59.4 |
| 12 | 12.0 | 1.15 | 13.8 | 10.4 | 60.1 | 49.7 | 49.5 | 59.1 | 59.1 |
| 16 | 12.0 | 1.14 | 13.7 | 10.5 | 60.5 | 50.1 | 49.9 | 59.4 | 59.3 |

### Comparative Embodiment 1

On a PET film Cosmoshine A4300 (film thickness: 250 μm, size: 130 mm×370 mm, adhesion layers are attached to opposite surfaces thereof, and an anti-fog coating film is formed at one surface thereof) manufactured by Toyobo Co., Ltd., as the transparent conductive film, an ITO film formed by performing sputtering in a vacuum state is used on the surface on which the anti-fog coating film is not formed, in which the ITO film is used instead of a silver lattice film. Then, other conditions were set to be same as Embodiment 1, and goggles for motorcycling were manufactured. Although there is a difference in the resistance values between the 50 sheets of the top and bottom electrodes, the resistance values were included between 9.0 Ω to 11.0 Ω and the average value of the resistance values was about 10.0 Ω and the average value of the surface resistance values was about 30 Ω/cm². One of the sheets was arbitrarily selected and the total light transmittance was measured. As a result, the total light transmittance was about 87% when the light transmittance of the PET substrate is excluded.

The haze value was 3.5 when the haze value of the PET substrate is included, and the ITO film was slightly colored in a light-yellow color. In the same manner as in Embodiment 1, goggles to which the transparent conductive film is attached was manufactured. The target reaching temperature of the goggles was 44° C.

The power source is connected to the goggles and the voltage of 12V was applied to the goggles so as to heat the goggles, and results were shown in Table 6. The non-contact type radiation thermometer (product name: fixed density radiation thermometer manufactured by Horiba Ltd., type: IT545S) was used to measure a temperature, and temperatures of several positions inside the goggles were measured. As a result, since a large difference in temperatures according to measurement positions was not identified, temperatures at the measurement position of the center portion of the goggles from the left and right positions and from the top and bottom positions of the goggles were recorded.

After four minutes, the temperature rose to 39 ° C., and, after eight minutes, the temperature reached about 44° C. that is the target reaching temperature. Under the environment in which an outside air temperature was −10° C. and a humidity was 40%, a helmet for snowmobiling on which the goggles were mounted was worn on a head, and the transparent conductive film was conductively heated by connecting the power source. As a result, when in a state in which the goggles were mounted on a face, there was no fog on the surface of lens, but the surface of the lens of the goggles was slightly colored in light-yellow color.

TABLE 6

| Elapsed time | Voltage | Current | Power | V/A | Temperature |
|---|---|---|---|---|---|
| (minutes) | (V) | (A) | (W) | (Ω) | (° C.) |
| 4 | 12.0 | 1.20 | 14.4 | 10.0 | 39.0 |
| 6 | 12.0 | 1.20 | 14.4 | 10.0 | 42.1 |
| 8 | 12.0 | 1.21 | 14.5 | 9.9 | 43.2 |
| 10 | 12.0 | 1.20 | 14.4 | 10.0 | 44.1 |
| 12 | 12.0 | 1.21 | 14.5 | 9.9 | 44.2 |
| 16 | 12.0 | 1.20 | 14.4 | 10.0 | 44.1 |

### Comparative Embodiment 2

Except that the lattice line width was changed from 20 μm to 25 μm, in the same condition as Embodiment 1, sheets of transparent heating elements were printed, dried, and fired. Although there is a difference in the resistance values between the 50 sheets of the top and bottom electrodes, the resistance values were included between 8.0 Ω to 9.5 Ω and the average value of the resistance values was about 8.8 Ω and the average value of the surface resistance values was about 26 Ω/cm². One of the sheets was arbitrarily selected and the total light transmittance was measured. As a result, the total light transmittance was about 93% when the light transmittance of the PET substrate is excluded. Since the lattice film does not cover the entire surface of the lens, the haze value of the lattice film was the same as the haze value of the PET substrate (the total light transmittance of the PET substrate A4300 having the thickness of 250 μm: 92.3%, haze value: 0.9%).

In the same manner that is used in Embodiment 1, goggles for snowmobiling were manufactured by arbitrarily selecting one sheet from these 50 sheets of the transparent heating elements formed of the silver lattice film that is printed, dried, and fired. The visibility when a helmet for snowmobiling on which the goggles were mounted was worn on a head was evaluated. As a result, when in a state in which the goggles were mounted on a face, the lines of the lattice pattern were perceived, and it has been confirmed that the transparent heating element in the goggles is not sufficient to be used since the sight of the goggles is interrupted.

### Comparative Embodiment 3

Except that the pitch having the lattice line width of 20 μm was changed from 1 μm to 5.5 μm, in the same condition as Embodiment 1, 50 sheets of transparent heating elements were printed, dried, and fired. One of the sheets was arbitrarily selected and the total light transmittance was measured. As a result, the total light transmittance was about 98% when the light transmittance of the PET substrate is excluded, but the average value of the surface resistance values was about 55 Ω/cm², so that it has been confirmed that it is not sufficient to be used as the transparent heating element since the resistance value is high.

### Comparative Embodiment 4

Except that the pitch having the lattice line width of 20 μm was changed from 1 μm to 0.5 μm, in the same condition as Embodiment 1, 50 sheets of transparent heating elements were printed, dried, and fired. One of the sheets was arbitrarily selected and the total light transmittance was measured. As a result, the total light transmittance was about 91% when the light transmittance of the PET substrate is excluded, but the average value of the surface resistance values was about 5 Ω/cm², so that it has been confirmed that it is not sufficient to be used as the transparent heating element since the resistance value is low.

### Comparative Embodiment 5

Except that the pitch having the lattice line width of 5 μm was changed from 100 μm to 70 μm, in the same condition as Embodiment 2, 50 sheets of transparent heating elements were printed, dried, and fired. One of the sheets was arbitrarily selected and the surface resistance values were measured. As a result, the average value of the surface resistance values was about 10 Ω/cm². However, since the total light transmittance was 87% when the light transmittance of the PET substrate is excluded, it has been confirmed that it is not sufficient to be used as the transparent heating element since the transparency is low.

Although the present disclosure invented by the present inventor has been described in detail with reference to the embodiments, the present disclosure is not limited to the above embodiments, and various modifications are possible without departing from the gist of the present disclosure.

### INDUSTRIAL APPLICABILITY

According to the transparent heating element of the present disclosure, the visibility of the goggles having the transparent base may be secured, and the transparent heating element having the dried material or the calcined material of the conductive ink or the conductive paste that is capable of being formed to have the desired resistance value may be provided, so that the transparent heating element is highly useful for goggles for snowmobiling, goggles for skiing, and goggles for motorcycling that require an anti-fog function.

1. A transparent heating element for an eye protector, the transparent heating element comprising:
a lattice film formed of a dried material or a calcined material of a conductive ink or a conductive paste and formed at at least one surface of a transparent base, the lattice film being configured such that a line width thereof is 2.5 μm to 20 μm, a lattice pitch thereof is 0.1 mm to 5.0 mm, a surface resistance value thereof is 10 Ω/cm² to 50 Ω/cm², and a light transmittance thereof excluding a portion of the transparent base is at least 90%,
wherein, in the lattice film, the surface resistance value of the surface on which the dried material or the calcined material of the conductive ink or the conductive paste is formed is partially changed by changing the lattice pitch.

2. The transparent heating element of claim 1, wherein the transparent base is an organic film or a glass base.

3. The transparent heating element of claim 1, wherein the transparent heating element is formed at one surface of the transparent base, and an anti-fog coating treatment is performed on opposite surface of the surface on which the transparent heating element is formed.

4. The transparent heating element of claim 1, wherein the eye protector is goggles for snowmobiling, goggles for skiing, or goggles for motorcycling.

5. A method of manufacturing the transparent heating element for an eye protector of claim 1, the method comprising: forming a lattice film of a conductive ink or a conductive paste by performing a screen printing method, a gravure printing method, a gravure offset printing method, a gravure reverse printing method, or an inkjet printing method.
